(19) United States
(12) Patent Application Publication
(10) Pub. No.: US 2006/0252074 A1
(43) Pub. Date: Nov. 9, 2006

(54) Immobilization and reaction matrix for biosensors

(76) Inventors: Melanie Atzesberger, Munchen (DE); Petra Schindler-Bauer, Vaterstetten (DE)

Correspondence Address: HARNESS, DICKEY & PIERCE, P.L.C., P.O.BOX 8910, RESTON, VA, 20195, US

(21) Appl. No.: 11/403,867

(22) Filed: Apr. 14, 2006

(30) Foreign Application Priority Data
Apr. 15, 2005 (DE) 10 2005 017 522.8

(51) Int. Cl. C12Q 1/68; C12M 1/34

(52) U.S. Cl. 435/6; 435/287.2

(57) ABSTRACT

An immobilization and reaction matrix based on a polymeric hydrogel for biosensor chips is disclosed, in particular those based on semiconductor chips which are disposed on a completely processed wafer and have, applied thereto, sensor fields which are normally disposed in array format. Such biosensor chips are normally functionalized by employing organic molecules such as, for example nucleic acids such as DNA, RNA, PNA and LNA or derivatives thereof, proteins, sugar molecules or antibodies.

The present application hereby claims priority under 35 U.S.C. §119 on German patent application number DE 10 2005 017 522.8 filed Apr. 15, 2005, the entire contents of which is hereby incorporated herein by reference.

### FIELD

The present invention generally relates to an immobilization and reaction matrix. For example, it may relate to one based on a polymeric hydrogel for biosensor chips, for example those based on semi-conductor chips which are disposed on a completely processed wafer and have, applied thereto, sensor fields which are normally disposed in array format. Such biosensor chips normally are functionalized by employing organic molecules such as, for example nucleic acids such as DNA, RNA, PNA and LNA or derivatives thereof, proteins, sugar molecules or antibodies.

### BACKGROUND

Application of capture molecules to biochips requires the use of biochemical “adhesion-promoting layers” or linkers. This is a process which is difficult to carry out and which often cannot be carried out in satisfactory quantity and quality.

The biochips to be coated are normally chemically pretreated in such a way that capture molecules are able to bind. The binding is, however, often unsatisfactory in quantity and quality. The electrical biochips are moreover frequently damaged or destroyed in the elaborate pretreatment. In addition, further processing must take place directly subsequently. This process is carried out manually, automation on the industrial scale not yet being possible. In addition, it is necessary to create a suitable reaction environment for carrying out the detection reaction, e.g. a hybridization, but this is usually insufficiently compatible with the coating chemistry.

Approaches using hydrogel as carrier matrix on an optically readable biochip already exist, although the composition of the gel is such that it has a denaturing effect on proteins, making an enzymatic reaction impossible, and such that a chemical linker is required and thus an equal distribution or increase in concentration of the molecules is impossible. Moreover, the reagents frequently include phosphates which have an inhibitory effect on redox cycling with para-aminophenyl phosphate, as further explained below.

### SUMMARY

At least one embodiment of the present invention is based on an object of providing a hydrogel which is suitable for functionalizing biochips and which firstly does not have a denaturing effect on proteins and secondly makes an increase in concentration of the capture molecules possible. Such a hydrogel ought further to be compatible with the biochip semiconductor surface, i.e. not enter into any reactions therewith. In particular, such a hydrogel ought also to ensure redox cycling as appropriate detection principle for a detection reaction, such as, for example, a hybridization, by the biosensor.

An object may be achieved by at least one of the embodiments discussed in the description below.

There is provided in particular a hydrogel-based immobilization and reaction matrix for biosensor chips, where the hydrogel comprises:
(i) a copolymerization reaction product of
   (a) a mixture of (meth)acrylamide and bis(meth)-acrylamide in the ratio from 20:1 to 45:1, based on weight, with a final concentration in the range from 3 to 15% by volume, based on the total volume of the hydrogel, and
   (b) Acrydite- or (meth)acrylamide-modified capture molecules in a concentration in the range from 0.001 pM to 100 mM, based on the total volume of the hydrogel, and
   (c) a polymerization initiator in a concentration in the range from 0.001% by volume to 10% by volume based on the total volume of the hydrogel,
      and where appropriate
(ii) conventional hydrogel additives.

The hydrogel of the invention preferably further comprises
(d) glycerol in a proportion of from 2 to 80% by volume, and
(e) tris(hydroxymethyl)aminomethane as buffer (“tris buffer”) in a concentration in the range from 1 mM to 1 M, based on the total volume of the hydrogel.

Conventional hydrogel additive refers to, in the context of the present application for example, complexing agents such as EDTA, surface-active agents such as SDS (sodium dodecyl sulfate), cations such as Na⁺ or Mg²⁺ to adjust the ion concentration, formamide, sugar derivatives such as dextrans or agarose, etc., whose incorporation into a hydrogel is within the routine judgment of a skilled worker.

The hydrogel of the present application additionally includes water, preferably double-distilled water, in conventional amounts.

The polymerization initiator preferably employed is ammonium persulfate in combination with N,N,N′,N′-tetramethylethylenediamine (TEMED).

(Meth)acrylamide refers to, in the context of the present application, both methacrylamides and acrylamides.

At least one embodiment of the present application further relates to a biosensor chip including a substrate having at least one semiconductor chip disposed thereon, with in turn at least one sensor field being disposed on the semiconductor chip and being present at the bottom of a cavity which is surrounded by a layer having a predetermined structure and which is applied completely to the entire surface of the semiconductor chip, with the hydrogel-based immobilization and reaction matrix of at least one embodiment of the invention being incorporated, as stated above, in the cavity above the sensor field. The sensor fields of the semiconductor chip are moreover may be designed, for example, as interdigitated electrode fields, preferably made of gold, platinum, titanium or palladium.

Functionalization and operation of biochips is facilitated on use of such hydrogel-based immobilization and reaction matrices of at least one embodiment of the invention through the capture molecules being incorporated into this specific hydrogel matrix, simultaneously resulting also in a suitable reaction environment for the hybridization.

At least one of the following advantages emerge from this, regarding at least one embodiment of the present application:
no elaborate chemical cleaning procedure is necessary before the coating,
the hydrogel of at least one embodiment of the invention represents a three-dimensional carrier layer in contrast to a two-dimensional application for example as SAM (self assembled monolayer),
higher concentrations of capture molecules can be immobilized and their distribution on the sensor is more uniform and denser,
the immobilization is very firm and undetachable,
preservation of the functional layer comprising the capture molecules is possible over a longer period,
accurately defined amounts of molecules can be applied,
the use of the hydrogel-based immobilization and reaction matrices of the invention is suitable both for active and passive or optical biochips,
loss of cohesion of the functional layer with subsequent crosstalk is prevented,
the use of the hydrogel-based immobilization and reaction matrix of the invention can be automated, also at the wafer level,
the reaction environment can be adjusted optimally as required in relation to various parameters such as pH, phosphate content, salt content, pore size, etc.,
the hydrogel of at least one embodiment of the invention is compatible with any surface,
the hydrogel of at least one embodiment of the invention is not denaturing for proteins, and no chemical linker is required for immobilizing the capture molecules, and the hydrogel of the invention is therefore suitable for nucleic acid chips, protein chips and cells on chips,
the mechanical properties and thermal stability are adequate.

The hydrogel of at least one embodiment of the invention can be introduced in particular initially in the liquid state into depressions on electrical biochips, as described in DE 10 2004 019357.6, the entire contents of which are incorporated herein by reference. The solution then includes a defined, predetermined concentration of appropriate capture molecules composed of nucleic acids (DNA, RNA, PNA, LNA) which are modified at one end by an Acrydite, a (meth)acrylamide or a derivative thereof.

The gel cures in the depression of the biochips and represents a gelatinous matrix for subsequent reactions. The capture molecules are virtually “embedded” in the gel, being polymerized via their Acrydite or (meth)acrylamide modification at one of the ends of the strand into the structure of the gel, i.e. copolymerized. This entails initially the Acrydite or (meth)acrylamide groups being introduced for example into the terminal 3′ or 5′ position of DNA molecules, followed by polymerization of such intermediates to form a hydrogel in which or on which capture molecules such as, for example, DNA molecules are covalently bonded. Such an Acrydite-modified DNA molecule is shown below: 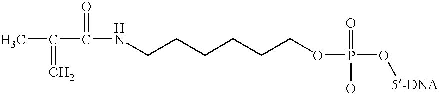
where the alkylene linker between phosphate and amide group can vary in the range from 4 C to 20 C atoms.

Instead of DNA molecules it is also possible to provide for example artificial antibodies or corresponding fragments as capture molecules, modified with Acrydite or (meth)acrylamide units.

The polymerization of the capture molecules inside the hydrogel matrix of at least one embodiment of the invention makes this type of linkage unbreakable under normal reaction conditions and stable toward environmental influences. It is thus possible for capture molecules to be introduced in equal distribution and in defined concentration into the matrix without the use of a chemical linker. The specific composition of the hydrogel allows a diffusion-driven exchange of reagents through the pores of the gel. The pore size can be specifically controlled and can be adjusted to the particular experimental conditions.

The composition of the hydrogel is designed so that it has no denaturing effect on proteins and thus makes enzymatic reactions possible. Detection by the method of redox cycling with alkaline phosphatase (ALP) is thus possible. The chemical properties of the gel make hybridization under optimized conditions possible. Unbound target molecules, ALP and/or substrate molecules can be washed out of the gel by washing buffers of defined stringency. Moreover, no phosphate ions having an inhibitory effect on ALP are introduced via reagents into the reaction.

The detection principle of redox cycling on biochips is known; cf. Hofmann et al, Passive DNA sensor with Gold Electrodes Fabricated in a CMOS Backend Process, ESSDERC 2002. This entails the need for different successive biochemical reactions requiring different reaction conditions to be carried out on the electrical chip.

Firstly capture molecules are immobilized, normally in the form of a tethering on the surface of gold electrodes employed by use of gold-thiol coupling. Subsequently, the chip is brought into contact with an analyte. This may entail hybridization of a target molecule (match) or just no hybridization (mismatch). This is followed for example by labeling on biotin, which is bound to the target molecule, with for example a streptavidin-coupled ALP.

A possible substrate suitable for use for starting the redox process is p-aminophenyl phosphate (p-APP). The resulting species from the enzymatic reaction initiated by ALP is para-aminophenol (p-AP). Then, on the electrodes of the chip, there is oxidation of para-aminophenol to quinone imine and reduction. The repeated p-aminophenol oxidation reaction and reduction reaction represent the actual “redox cycling”.

All the reactions taking place must take place under conditions which are as optimal as possible, but these must not have a negative effect on the other reactions and/or damage the electrical chip or its surface. This is ensured on use of the hydrogel-based immobilization and reaction matrix of at least one embodiment of the invention instead of immobilization of capture molecules by means of gold-thiol coupling in the form of SAMs, i.e. the redox cycling is not negatively affected. In addition, the hydrogel-based immobilization and reaction matrix of at least one embodiment of the invention also makes it possible to increase the concentration of the capture molecules.

### DETAILED DESCRIPTION OF THE EXAMPLE EMBODIMENTS

An embodiment of the present invention is explained in more detail by the following example, without being restricted thereto.

Firstly a photopatternable coating (e.g. SU-8) is applied to the surface of a biosensor chip and, after illumination, forms a type of “well” around the individual sensor fields; cf. DE 10 2004 019357.6. The sensor fields thus form the base of a vessel with a capacity of, typically, 1 nl. The walls of the vessel are typically 80 μm high.

A liquid hydrogel including a mixture of acrylamide and bisacrylamide (typically in the ratio 37.5:1), glycerol, tris base (0.4 M, pH 8-11), water and Acrydite-modified DNA capture molecules is degassed and, after addition of ammonium persulfate and TEMED as initiator, put into the wells for the polymerization. Alternatively, it is also possible initially to put only part of the mixture thereof into the wells, followed by addition of one or more of the remaining constituents necessary to form the hydrogel of at least one embodiment of the invention. It is moreover possible to put a different species (and/or concentration) of capture molecules in each well.

The polymerization in the wells takes place faster or slower, depending on the temperature and the total concentration of acrylamides and initiator. After the polymerization has taken place, the gel is so viscous that it stays firmly in the vessels, and not even rinsing the pores of the gel with the washing or reaction liquids influences the mechanical stability.

If a sheet (e.g. dicing tape) is stuck over the chip at this time, the contents of the wells together with the capture molecules are effectively protected from external influences and also evaporation, so that the possibilities for manipulating the chip (e.g. sawing) and the period of storage of the functionalized chips increase many-fold.

If a chip preserved in this way is used for detection, the sheet can be detached without residues after UV irradiation (e.g. 20 s at 318 nm) and heating at 95° C. The properties of the gel are not altered by this process.

The “target” DNA molecules to be hybridized are labeled for example with biotin. They are typically put in tris base buffer on top of the wells and diffuse at different rates, depending on the length of the DNA molecule, the pore size of the gel and the temperature, into the gel matrix and, where appropriate, hybridize with a complementary capture molecule strand.

Several washing steps with buffer in which the non-hybridized target molecules are removed are followed by covering the reaction mixture with a layer of an appropriately diluted alkaline phosphatase which—e.g. recombinantly—is coupled to an Extravidin or streptavidin. The ALP diffuses as a function of temperature and pore size into the gel matrix and binds to the biotin marker of the hybridized target DNA.

Further washing steps with buffer in which the unbound enzymes are removed can be followed by addition of the diluted enzyme substrate p-aminophenyl phosphate. Care must be taken that non-enzymatic hydrolysis of the substrate is reduced as far as possible or even minimized, by using or even optimizing the hydrogel composition and the reaction conditions. The reaction of the substrate then takes place by enzymatic elimination of a phosphate group to give p-aminophenol and oxidation and reduction thereof on the finger-like electrodes of the respective sensor field. The redox signal is detected in a suitable apparatus.

An increased selectivity (stringency) of hybridization can be achieved if the washing steps after the hybridization are optimized. The stringency depends inter alia on the length of the DNA, the GC content in percent, the length and location of possible mismatches, the salt concentration, the temperature and the pH. A stringency treatment must be carried out in such a way that correctly hybridized bindings are not damaged.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A hydrogel-based immobilization and reaction matrix for biosensor chips, the hydrogel comprising:
(i) a copolymerization reaction product of
(a) a mixture of (meth)acrylamide and bis-(meth)acrylamide in the ratio from 20:1 to 45:1, based on weight, with a final concentration in the range from 3 to 15% by volume, based on the total volume of the hydrogel, and
(b) Acrydite- or (meth)acrylamide-modified capture molecules in a concentration in the range from 0.001 pM to 100 mM, based on the total volume of the hydrogel, and
(c) a polymerization initiator in a concentration in the range from 0.001% by volume to 10% by volume based on the total volume of the hydrogel,
and where appropriate
(ii) conventional hydrogel additives.

2. The hydrogel-based immobilization and reaction matrix for biosensor chips as claimed in claim 1, further comprising
(d) glycerol in a proportion of from 2 to 80% by volume, and
(e) tris(hydroxymethyl)aminomethane as buffer in a concentration in the range from 1 mM to 1 M, based on the total volume of the hydrogel.

3. The hydrogel-based immobilization and reaction matrix for biosensor chips as claimed in claim 1, wherein the polymerization initiator is based on ammonium persulfate in combination with N,N,N′,N′-tetramethylethylenediamine (TEMED).

4. The hydrogel-based immobilization and reaction matrix for biosensor chips as claimed in claim 1, wherein the biosensor chip is a CMOS-based semiconductor chip.

5. The hydrogel-based immobilization and reaction matrix for biosensor chips as claimed in claim 1, wherein the capture molecules are selected from nucleic acids such as DNA, RNA, PNA and LNA or derivatives thereof, proteins, sugar molecules or antibodies and are preferably oligonucleotide probes.

6. A biosensor chip, comprising:
a substrate having at least one semiconductor chip disposed thereon, with in turn at least one sensor field being disposed on the semiconductor chip and being present at the bottom of a cavity which is surrounded by a layer having a predetermined structure and which is applied completely to the entire surface of the semiconductor chip, with the hydrogel-based immobilization and reaction matrix, as defined in claim 1, being incorporated in the cavity above the sensor field.

7. The biosensor chip as claimed in claim 6, wherein the sensor fields of the semiconductor chip are designed as interdigitated electrode fields.

8. The hydrogel-based immobilization and reaction matrix for biosensor chips as claimed in claim 2, wherein the polymerization initiator is based on ammonium persulfate in combination with N,N,N′,N′-tetramethylethylenediamine (TEMED).

9. The hydrogel-based immobilization and reaction matrix for biosensor chips as claimed in claim 2, wherein the biosensor chip is a CMOS-based semiconductor chip.

10. The hydrogel-based immobilization and reaction matrix for biosensor chips as claimed in claim 3, wherein the biosensor chip is a CMOS-based semiconductor chip.

11. The hydrogel-based immobilization and reaction matrix for biosensor chips as claimed in claim 8, wherein the biosensor chip is a CMOS-based semiconductor chip.

12. The hydrogel-based immobilization and reaction matrix for biosensor chips as claimed in claim 2, wherein the capture molecules are selected from nucleic acids such as DNA, RNA, PNA and LNA or derivatives thereof, proteins, sugar molecules or antibodies and are preferably oligonucleotide probes.

13. The hydrogel-based immobilization and reaction matrix for biosensor chips as claimed in claim 3, wherein the capture molecules are selected from nucleic acids such as DNA, RNA, PNA and LNA or derivatives thereof, proteins, sugar molecules or antibodies and are preferably oligonucleotide probes.

14. The hydrogel-based immobilization and reaction matrix for biosensor chips as claimed in claim 4, wherein the capture molecules are selected from nucleic acids such as DNA, RNA, PNA and LNA or derivatives thereof, proteins, sugar molecules or antibodies and are preferably oligonucleotide probes.

15. A biosensor chip, comprising:
a substrate having at least one semiconductor chip disposed thereon, with in turn at least one sensor field being disposed on the semiconductor chip and being present at the bottom of a cavity which is surrounded by a layer having a predetermined structure and which is applied completely to the entire surface of the semiconductor chip, with the hydrogel-based immobilization and reaction matrix, as defined in claim 2, being incorporated in the cavity above the sensor field.

16. A biosensor chip, comprising:
a substrate having at least one semiconductor chip disposed thereon, with in turn at least one sensor field being disposed on the semiconductor chip and being present at the bottom of a cavity which is surrounded by a layer having a predetermined structure and which is applied completely to the entire surface of the semiconductor chip, with the hydrogel-based immobilization and reaction matrix, as defined in claim 3, being incorporated in the cavity above the sensor field.

17. A biosensor chip, comprising:
a substrate having at least one semiconductor chip disposed thereon, with in turn at least one sensor field being disposed on the semiconductor chip and being present at the bottom of a cavity which is surrounded by a layer having a predetermined structure and which is applied completely to the entire surface of the semiconductor chip, with the hydrogel-based immobilization and reaction matrix, as defined in claim 4, being incorporated in the cavity above the sensor field.

18. A biosensor chip, comprising:
a substrate having at least one semiconductor chip disposed thereon, with in turn at least one sensor field being disposed on the semiconductor chip and being present at the bottom of a cavity which is surrounded by a layer having a predetermined structure and which is applied completely to the entire surface of the semiconductor chip, with the hydrogel-based immobilization and reaction matrix, as defined in claim 5, being incorporated in the cavity above the sensor field.

19. The biosensor chip as claimed in claim 6, wherein the sensor fields of the semiconductor chip are designed as interdigitated electrode fields, made of at least one of gold, platinum, titanium and palladium.
